(19) United States
(12) Patent Application Publication
(10) Pub. No.: US 2017/0355729 A1
(43) Pub. Date: Dec. 14, 2017

(54) METHODS AND COMPOSITIONS FOR CONTROLLING ANTIBODY AGGREGATION

(71) Applicant: Biogen MA Inc., Cambridge, MA (US)

(72) Inventors: Matthew Westoby, Chapel Hill, NC (US); Alex Brinkmann, Cary, NC (US); Ryan Haverstock, Encinitas, CA (US); Roy Alston, Framingham, MA (US)

(73) Assignee: Biogen MA Inc., Cambridge, MA (US)

(21) Appl. No.: 15/532,578

(22) PCT Filed: Dec. 2, 2015
(86) PCT No.: PCT/US2015/063339

§ 371 Date: Jun. 2, 2017

Related U.S. Application Data
(60) Provisional application No. 62/086,629, filed on Dec. 2, 2014.

(51) Int. Cl. C07K 1/34; C07K 16/24; A61K 39/395

(52) U.S. Cl. CPC C07K 1/34; A61K 39/39591; C07K 16/241; C07K 2317/24

(57) ABSTRACT

Disclosed herein are methods of purifying monoclonal antibodies and related compositions.

### RELATED APPLICATION

This application claims the benefit under 35 U.S.C. §119(e) of U.S. provisional application number 62/086,629, filed Dec. 2, 2014, which is incorporated by reference herein in its entirety.

### FIELD OF THE INVENTION

The present disclosure relates to the field of protein purification.

### BACKGROUND OF INVENTION

Protein aggregation is frequently observed at several stages of bioprocessing, including protein expression, purification and storage. Protein aggregation can affect the overall yield of therapeutic protein (e.g., antibody) manufacturing processes and may contribute to stability and immunogenicity of therapeutic proteins.

### SUMMARY OF INVENTION

The present disclosure provides methods and compositions for antibody purification and storage that reduce antibody aggregation and enhance antibody stability. The present disclosure is based, in part, on results showing that significant protein aggregation occurs during bioprocessing of a commercial-based infliximab monoclonal antibody composition in a buffer having a pH of 7.2 (e.g., in a 10 mM sodium phosphate and 10% sucrose buffer). The present disclosure provides methods for reducing antibody aggregation by processing the antibody (e.g., by ultrafiltration/diafiltration) at a pH of less than 7.2, and in some instances, at a temperature of 2° C. to 12° C.

In some embodiments, a reduction in protein aggregation is achieved during bioprocessing of infliximab (1) by reducing the sodium phosphate concentration and lowering the pH of the antibody composition during filtration (e.g., ultrafiltration/diafiltration) to a pH of less than 7.2 (e.g., pH of 5.7 to 6.7), and then increasing the pH (e.g., to 7.2) following filtration, or (2) by filtering the antibody composition having a pH of 7.2 into a buffer containing 10 mM sodium phosphate and having a pH of less than 7.2 (e.g., pH of 5.7 to 6.7), with or without sucrose.

In some embodiments, a reduction in protein aggregation is achieved during bioprocessing of infliximab by reducing the temperature (e.g., less than 12° C.) of the processing conditions.

Unexpectedly, the aqueous infliximab compositions provided herein (e.g., comprising 10 mM sodium phosphate, 10% sucrose), which have a pH of less than 7.2 (e.g., pH of 5.7 to 6.4), are stable for a longer period of time relative to existing commercial-based compositions.

Some embodiments of the present disclosure provide antibody processing methods, comprising filtering through a membrane filter a phosphate buffered antibody solution having a pH of 5.7 to 6.7 and comprising infliximab, thereby producing an antibody concentrate, wherein antibody aggregation in the phosphate buffered antibody solution is reduced relative to a control antibody solution comprising infliximab and having a pH of 7.2.

Some embodiments of the present disclosure provide compositions comprising an antibody solution comprising infliximab and having a pH of 5.7 to 6.7. In some embodiments, the infliximab is more stable when the composition is stored at temperatures of −70° C. to at least 40° C. relative to a control composition having a pH of 7.2.

In some embodiments, an antibody solution has a pH of 5.7 to 6.7, 5.8 to 6.6, 5.9 to 6.5, 6.0 to 6.4, or 6.1 to 6.3. For example, an antibody solution may have a pH of 5.7, 5.8, 5.9, 6.0, 6.1, 6.2, 6.3, 6.4, 6.5, 6.6 or 6.7.

In some embodiments, an antibody solution comprises sodium phosphate. In some embodiments, the concentration of the sodium phosphate in an antibody solution is 5 mM to 15 mM. For example, the concentration of the sodium phosphate in an antibody solution may be 10 mM. In some embodiments, the concentration of the sodium phosphate in an antibody solution is 6 mM to 14 mM, 7 mM to 13 mM, 8 mM to 12 mM, or 9 mM to 10 mM.

In some embodiments, an antibody solution comprises sucrose. In some embodiments, the concentration of sucrose in an antibody solution is 5% to 15% (w/v). For example, the concentration of sucrose in an antibody solution may be 10% (w/v). In some embodiments, the concentration of sucrose in an antibody solution is 6% to 14% (w/v), 7% to 13% (w/v), 8% to 12% (w/v), or 9% to 11% (w/v).

In some embodiments, an antibody solution comprises a surfactant. In some embodiments, the concentration of surfactant in an antibody solution is 0.005% to 0.015% (w/v). For example, the concentration of surfactant in an antibody solution may be 0.01% (w/v). In some embodiments, the surfactant is polysorbate 80. In some embodiments, the concentration of surfactant in an antibody solution is 0.006% to 0.014% (w/v), 0.007% to 0.013% (w/v), 0.008% to 0.012% (w/v), or 0.009% to 0.011% (w/v).

In some embodiments, an antibody solution comprises infliximab at a concentration of 15 g/L to 100 g/L. For example, an antibody solution may comprise infliximab at a concentration of or 20 g/L to 95 g/L, 25 g/L to 90 g/L, 30 g/L to 85 g/L, 35 g/L to 80 g/L, 40 g/L to 75 g/L, 45 g/L to 70 g/L, or 50 g/L to 65 g/L. In some embodiments, an antibody solution comprises infliximab at a concentration of 20 g/L to 60 g/L.

In some embodiments, the temperature of the antibody solution is less than room temperature (e.g., 25° C.). In some embodiments, the temperature of the antibody solution is 2° C. to 12° C. For example, the temperature of the antibody solution may be 4° C. to 6° C. In some embodiments, the temperature of the antibody solution is 3° C. to 11° C., 4° C. to 10° C., 5° C. to 9° C., or 4° C. to 8° C. In some embodiments, the temperature of the antibody solution is 2° C., 3° C., 4° C., 5° C., 6° C., 7° C., 8° C., 9° C., 10° C., 11° C. or 12° C.

In some embodiments, a membrane filter has a pore size of 0.1 to 0.001 micron. For example, a membrane filter may have a pore size of 0.09 to 0.002 micron, 0.08 to 0.003 micron, 0.07 to 0.004 micron, 0.06 to 0.005 micron, 0.05 to 0.006 micron, 0.04 to 0.007 micron, 0.03 to 0.008 micron, or 0.02 to 0.009 micron.

In some embodiments, an antibody solution is filtered through the membrane filter by tangential flow filtration.

In some embodiments, methods further comprise collecting an antibody concentrate.

### BRIEF DESCRIPTION OF DRAWINGS

FIG. 1 shows a graph of results obtained from a size exclusion chromatography (SEC) analysis of flow-through product collected at different stages of the ultrafiltration/diafiltration (UF/DF) operation described in Example 1. The graph compares changes in the percentage of high molecular weight species present during different bioprocessing steps.

FIG. 2 shows a graph of results obtained from a SEC analysis of dialysis products from experiments described in Example 2 (Phos=10 mM sodium phosphate, suc=10% sucrose, PS80=0.01% polysorbate 80). Experiments not designated “2-8° C.” were performed at ambient temperature. HIC-FT: hydrophobic interaction chromatography-flow-through.

FIG. 3 shows a graph of pH of sample solutions collected at various intervals during a UF/DF operation performed using modified diafiltration buffers, as described in Example 3.

FIG. 4 shows a graph of pH of sample solutions collected at various intervals during a UF/DF operation performed using modified diafiltration buffers, as described in Example 3.

FIG. 5 shows a graph of the results of a SEC analysis, as described in Example 4, comparing change in percentage of high molecular weight species formation from the first small scale UF/DF run from a prototype bioprocessing run (PT-02).

FIG. 6 shows a graph of the results obtained from a SEC analysis comparing change in percentage of high molecular weight species formation at various stages of the UF/DF operation for two batches of the antibody, as described in Example 5.

FIG. 7 shows a graph of the results obtained from a SEC analysis at various stages of the UF/DF operation, as described in Example 5.

FIG. 8 shows a work flow of the UF/DF operation beginning with the phenyl sepharose flow-through with an antibody concentration of 2.2 g/L and resulting in various antibody compositions, as described in Example 6. The percentage of high molecular weight species formation at each stage of the operation is presented in boldface. The composition, pH and antibody concentration are also presented for the antibody solutions at each stage.

FIG. 9 shows a work flow of the UF/DF operation beginning with phenyl sepharose flow-through with an antibody concentration of 2.3 g/L and resulting in various antibody compositions, as described in Example 6. The percentage of high molecular weight species formation at each stage of the operation is presented in boldface. The composition, pH and antibody concentration are also presented for the antibody solutions at each stage.

FIG. 10 shows a graph of the percentage of aggregates (high molecular weight species) obtained from a SEC analysis of antibody solutions at various pH values prior to (TO) or after agitation (Agit) or cycles of freeze/thaw (FT), as described in Example 7.

FIG. 11 shows a graph of the percentage of aggregates (high molecular weight species) obtained from a SEC analysis of antibody solutions at various pH values prior to (TO) or after incubation of the antibody solutions for 2 weeks at 5° C., 25° C. or 40° C., as described in Example 7.

### DETAILED DESCRIPTION OF INVENTION

Provided herein are methods for purifying therapeutic proteins, such as therapeutic antibodies (e.g., chimeric monoclonal antibodies, such as infliximab), and compositions comprising such therapeutic proteins. Methods of the present disclosure are based, in part, on experimental evidence showing that potentially detrimental protein aggregation occurs during bioprocessing of a commercial-based infliximab antibody composition having a pH of 7.2. The present disclosure shows that reducing the pH of the commercial-based antibody composition, and in some instances, the temperature of the bioprocessing conditions, has a surprisingly positive effect on protein aggregation and protein stability (reducing protein aggregation and enhancing protein stability).

Manufacturing therapeutic antibodies requires a bioprocessing technique, referred to as diafiltration, which uses an ultrafiltration membrane filter (e.g., with pore sizes in the range of 0.1 to 0.001 micron) to remove, replace or lower the concentration of salts or solvents from solutions containing the antibodies. The filtration process selectively utilizes permeable (e.g., porous) membranes to separate the components of solutions and suspensions based on their molecular size. An ultrafiltration membrane, for example, retains molecules that are larger than the pores of the membrane while smaller molecules such as salts, solvents and water, which are 100% permeable, freely pass through the membrane. The solution retained by the membrane is referred to as a “concentrate” or “retentate,” while the solution that passes through the membrane is referred to as a “filtrate” or “permeate.” Thus, an “antibody concentrate” herein refers to a concentrated antibody solution that is retained by the membrane.

Tangential flow filtration, also referred to as crossflow filtration, refers to the mode by which a solution is passed tangentially across the surface of a membrane, rather than passed through a membrane, whereby the solids (or retentate) is collected by the membrane. By comparison, dead-end filtration refers to the mode by which a solution is passed through a membrane. One advantage of tangential flow filtration is that the filter cake (which can blind the filter) is substantially washed away during the filtration process, increasing the length of time that a filter unit can be operational. Tangential flow filtration, dead-end filtration and other modes of filtration are contemplated herein.

Membrane filters, such as ultrafiltration membranes, of the present disclosure may have a pore size of 0.001 to 0.1 micron. In some embodiments, a membrane filter has a pore size of 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095 or 0.100 micron. In some embodiments, ultrafiltration membrane are used for tangential flow filtration, dead-end filtration and/or other modes of filtration.

In some embodiments, membrane filters of the present disclosure have a molecular cut-off value of 15 kilodaltons (kDa) to 50 kDa, or more. For example, in some embodiments, a membrane filter has a molecular cut-off value of 15 kDa, 20 kDa, 25 kDa, 30 kDa, 35 kDa, 40 kDa, 45 kDa or 50 kDa, or any intermediate value. In some embodiments, the molecular weight cut off of the membrane is 30 kDa. In some embodiments, membrane filters having a molecular cut-off value of 15 kilodaltons (kDa) to 50 kDa, or more, are used for tangential flow filtration, dead-end filtration and/or other modes of filtration.

In some embodiments, membranes used herein to purify and/or concentrate an antibody solution (e.g., a buffered solution containing an antibody) are hydrophilic membranes (e.g., polyethersulfone (PES) membranes). In some embodiments, membranes are cellulose membranes, such as, for example, cellulose acetate membranes. In some embodiments, membranes are polyvinylidene fluoride (PVDF) membranes.

In some embodiments, the ultrafiltration membrane is a cellulose membrane with a molecular weight cut-off of 30 kDa. In some embodiments, the ultrafiltration membrane is an EKV PES membrane (e.g., Pall SUPOR® filter) with a molecular weight cut-off of 30 kDa.

In some embodiments, the solution may be pre-filtered through a membrane prior to applying the solution to an ultrafiltration membrane to reduce clogging of the ultrafiltration membrane.

A “therapeutic protein” refers to a protein that can be used to treat a condition. Examples of therapeutic proteins include antibodies and antibody fragments (e.g., a Fab fragment, or a Fc fragment). Antibodies and antibody fragments of the present disclosure, in some embodiments, are non-naturally-occurring (e.g., monoclonal antibodies, chimeric antibodies, recombinant antibodies , and/or humanized antibodies).

In some embodiments, antibodies of the present disclosure are monoclonal antibodies. “Monoclonal antibodies” are monospecific antibodies that are identical to each other and bind to the same epitope. Fragments of monoclonal antibodies are contemplated herein. In some embodiments, monoclonal antibodies are obtained from a recombinant cell line, such as a hybridoma.

In some embodiments, antibodies of the present disclosure are chimeric antibodies. “Chimeric antibodies” are antibodies having a mixture of components from different species (e.g., a mixture of non-human (e.g., mouse, rabbit, rat) and human components). In some embodiments, chimeric antibodies retain the binding specificity of the antibody of a non-human species but are expected to have reduced immunogenicity when administered to a human subject. Fragments of chimeric antibodies are contemplated herein. Also contemplated herein are chimeric monoclonal antibodies and fragments thereof.

In some embodiments, a chimeric monoclonal antibody of the present disclosure is infliximab (REMICADE®). Infliximab is a chimeric monoclonal antibody (chimeric IgG1) against (e.g., that binds specifically to) tumor necrosis factor alpha (TNF-α) and is typically used to treat, for example, autoimmune diseases. Infliximab includes human constant and murine variable regions and is produced by a recombinant cell line cultured by continuous perfusion.

Antibody (e.g., infliximab) compositions of the present disclosure are typically formulated in a phosphate buffered solution having a pH of less than 7.2. In some embodiments, a phosphate buffered solution comprises sodium phosphate. In some embodiments, the concentration of sodium phosphate in a phosphate buffered solution is 1 mM to 50 mM, 5 mM to 25 mM, 5 mM to 15 mM, or 5 mM to 10 mM. In some embodiments, the concentration of sodium phosphate in a phosphate buffered solution is 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM or 50 mM, or any intermediate value. In some embodiments, a phosphate buffered solution comprises sodium phosphate at a concentration of 10 mM. In some embodiments, a phosphate buffered solution comprises sodium phosphate at a concentration of 9 mM to 11 mM, 8 mM to 12 mM, or 7 mM to 13 mM. In some embodiments, a composition comprises a phosphate buffered solution that includes sodium phosphate at a concentration of 10 mM (or 9 mM to 11 mM, or 8 mM to 12 mM) and an antibody (e.g., infliximab).

In some embodiments, a composition comprises sucrose. In some embodiments, the concentration of sucrose in a composition is 1% to 30%, 5% to 20%, 5% to 15%, or 5% to 10% weight/volume (w/v). In some embodiments, the concentration of sucrose in a composition is 1% w/v, 2% w/v, 3% w/v, 4% w/v, 5% w/v, 6% w/v, 7% w/v, 8% w/v, 9% w/v, 10% w/v, 11% w/v, 12% w/v, 13% w/v, 14% w/v, 15% w/v, 16% w/v, 17% w/v, 18% w/v, 19% w/v, 20% w/v, 25% w/v or 30% w/v, or any intermediate value. In some embodiments, a composition comprises sucrose at a concentration of 10% w/v. In some embodiments, a composition comprises sucrose at a concentration of 9% w/v to 11% w/v, 8% w/v to 12% w/v, or 7% w/v to 13% w/v. In some embodiments, a composition comprises a phosphate buffered solution that includes sucrose at a concentration of 10% w/v (or 9% w/v to 11% w/v, or 8% w/v to 12% w/v) and an antibody (e.g., infliximab). In some embodiments, a composition comprises a phosphate buffered solution that comprises sodium phosphate at a concentration of 10 mM, sucrose at a concentration of 10% w/v (or 9% w/v to 11% w/v, or 8% w/v to 12% w/v) and an antibody (e.g., infliximab).

In some embodiments, a composition comprises a surfactant. In some embodiments, the concentration of surfactant in a composition is 0.001% to 0.030% w/v, 0.001% to 0.020% w/v, 0.005% to 0.015% w/v, or 0.005% to 0.025% w/v. In some embodiments, the concentration of surfactant in a composition is 0.001% w/v, 0.002% w/v, 0.003% w/v, 0.004% w/v, 0.005% w/v, 0.006% w/v, 0.007% w/v, 0.008% w/v, 0.009% w/v, 0.010% w/v, 0.011% w/v, 0.012% w/v, 0.013% w/v, 0.014% w/v, 0.015% w/v, 0.016% w/v, 0.017% w/v, 0.018% w/v, 0.019% w/v, 0.020% w/v, 0.025% w/v or 0.030% w/v, or any intermediate value. In some embodiments, a composition comprises surfactant at a concentration of 0.01% w/v. In some embodiments, a composition comprises surfactant at a concentration of 0.009% w/v to 0.02% w/v, 0.008% w/v to 0.03% w/v, or 0.007% w/v to 0.04% w/v. In some embodiments, a composition comprises surfactant at a concentration of 0.01% w/v (or 0.009% w/v to 0.02% w/v, or 0.008% w/v to 0.03% w/v) and an antibody (e.g., infliximab). In some embodiments, a composition comprises a phosphate buffered solution that includes sodium phosphate at a concentration of 10 mM, sucrose at a concentration of 10% w/v (or 9% w/v to 11% w/v, or 8% w/v to 12% w/v), surfactant at a concentration of 0.01% w/v (or 0.009% w/v to 0.02% w/v, or 0.008% w/v to 0.03% w/v) and an antibody (e.g., infliximab).

Examples of surfactants for use in accordance with the present disclosure include, without limitation, polyoxyethylene glycol sorbitan alkyl esters (e.g., polysorbate), fatty alcohols, cetyl alcohol, stearyl alcohol, cetostearyl alcohol, oleyl alcohol, polyoxyethylene glycol alkyl ether, polyoxypropylene glycol alkyl ether, glucoside alkyl ethers, polyoxyethylene glycol octylphenol ethers, polyoxyethylene glycol alkylphenol ethers, glycerol alkyl esters, olyoxyethylene glycol sorbitan alkyl esters, sorbitan alkyl esters, cocamide MEA, cocamide DEA, dodecyldimethylamine oxide, block copolymers of polyethylene glycol and polypropylene glycol and polyethoxylated tallow amine. In some embodiments, a phosphate buffered solution comprises polysorbate, e.g., polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), or polysorbate 80 (polyoxyethylene (20) sorbitan monooleate). In some embodiments, a phosphate buffered solution comprises polysorbate 80.

In some embodiments, a composition comprises polysorbate 80 at a concentration of 0.01% w/v (or 0.009% w/v to 0.02% w/v, or 0.008% w/v to 0.03% w/v). In some embodiments, a composition comprises polysorbate 80 at a concentration of 0.01% w/v (or 0.009% w/v to 0.02% w/v, or 0.008% w/v to 0.03% w/v) and an antibody (e.g., infliximab). In some embodiments, a composition comprises a phosphate buffered solution that includes sodium phosphate at a concentration of 10 mM (or 9 mM to 11 mM, or 8 mM to 12 mM), sucrose at a concentration of 10% w/v (or 9% w/v to 11% w/v, or 8% w/v to 12% w/v), polysorbate 80 at a concentration of 0.01% w/v (or 0.009% w/v to 0.02% w/v, or 0.008% w/v to 0.03% w/v) and an antibody (e.g., infliximab).

In some embodiments, a composition has a pH of less of than 7.2 (e.g., pH 5.5 to pH 7.1). In some embodiments, a composition has a pH of less than 7.1, less than 7.0, less than 6.9, less than 6.8, less than 6.7, less than 6.6, less than 6.5, less than 6.4, less than 6.3, less than 6.2, less than 6.1, less than 6.0, less than 5.9, less than 5.8, less than 5.7, or less than 5.6. In some embodiments, a composition has a pH of less than or equal to 7.1, 7.0, 6.9, 6.8, 6.7, 6.6, 6.5, 6.4, 6.3, 6.2, 6.1, 6.0, 5.9, 5.8, or 5.7. In some embodiments, a composition has a pH of less than or equal to 6.2. In some embodiments, a composition has a pH of greater than 5.5 and less than 7.2. In some embodiments, a composition has a pH of greater than 5.6 and less than 7.1, greater than 5.7 and less than 7.0, greater than 5.8 and less than 6.9, greater than 5.9 and less than 6.8, greater than 6.0 and less than 6.7, or greater than 6.1 and less than 6.6. In some embodiments, a composition has a pH of 5.5 to 7.1, 5.6 to 7.0, 5.7 to 6.9, 5.8 to 6.8, 5.9 to 6.7, 6.0 to 6.6, or 6.1 to 6.5. In some embodiments, a composition has a pH of 5.7 to 6.7. In some embodiments, a composition comprises a phosphate buffered solution that includes sodium phosphate at a concentration of 10 mM (or 9 mM to 11 mM, or 8 mM to 12 mM), optionally sucrose at a concentration of 10% w/v (or 9% w/v to 11% w/v, or 8% w/v to 12% w/v), optionally polysorbate 80 at a concentration of 0.01% w/v (or 0.009% w/v to 0.02% w/v, or 0.008% w/v to 0.03% w/v), an antibody (e.g., infliximab), and has a pH of less than 7.2, less than 7.1, less than 7.0, less than 6.9, or less than 6.8.

In some embodiments, a composition has a pH of 6.2. In some embodiments, a composition has a pH of 6.1 to 6.3, or 6.0 to 6.4. In some embodiments, a composition has a pH of 6.2 (or a pH of 6.1 to 6.3, or 6.0 to 6.4) and comprises an antibody (e.g., infliximab). In some embodiments, a comprises a phosphate buffered solution that includes sodium phosphate at a concentration of 10 mM (or 9 mM to 11 mM, or 8 mM to 10 mM or 8 mM to 12 mM), optionally sucrose at a concentration of 10% w/v (or 9% w/v to 11% w/v, or 8% w/v to 12% w/v), optionally polysorbate 80 at a concentration of 0.01% w/v (or 0.009% w/v to 0.02% w/v, or 0.008% w/v to 0.03% w/v), an antibody (e.g., infliximab), and has a pH of 6.2 (or a pH of 6.1 to 6.3, or 6.0 to 6.4).

In some embodiments, a composition comprises an antibody (e.g., infliximab) at a concentration of 10 g/L to 200 g/L, 15 g/L to 100 g/L, or 20 g/L to 60 g/L. In some embodiments, the concentration of the antibody is 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 26 g/L, 27 g/L, 28 g/L, 29 g/L, 30 g/L, 50 g/L, 75 g/L, 100 g/L or 200 g/L, or any intermediate value. In some embodiments, the concentration of an antibody in a composition is 20 g/L (or 19 g/L to 21 g/L, or 18 g/L to 22 g/L).

In some embodiments, a composition comprises an antibody (e.g., infliximab), 10 mM sodium phosphate, 10% sucrose and has a pH of 5.7 to 6.4.

In some embodiments, the aggregation of an antibody in a composition may be reduced by maintaining the composition, or the buffer in the composition, at reduced temperature (e.g., 2° C. to 12° C.), or performing the filtration at a reduced temperature. In some embodiments, a composition or buffer has a temperature of 1° C. to 20° C., 2° C. to 15° C., 2° C. to 12° C., 3° C. to 10° C., 4° C. to 8° C., 4° C. to 6° C., or 4° C. to 5° C. In some embodiments, a composition or buffer has a temperature of 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C. or 20° C., or any intermediate value. In some embodiments, a composition or buffer has a temperature of 2° C. to 12° C. In some embodiments, a composition or buffer has a temperature of 4° C. to 6° C.

Methods described herein, in some embodiments, result in production of an antibody (e.g., infliximab) with improved properties as compared to a control composition comprising an antibody (e.g., infliximab) and having a pH of 7.2. In some embodiments, antibody aggregation is reduced in a composition of the present disclosure relative to a control composition comprising the antibody (e.g., infliximab) and having a pH of 7.2. In some embodiments, the antibody aggregation in the composition is reduced by at least 10%, 20%, 30%, 40%, 50%, 60%, 70%, or at least 80% relative to a control composition comprising the antibody (e.g., infliximab). In some embodiments, the antibody aggregation in the composition is reduced by at least 10%, 20%, 30%, 40%, 50%, 60%, 70%, or at least 80% relative to a control composition comprising the antibody (e.g., infliximab). In some embodiments, a composition has reduced antibody aggregation when stored at 5° C. to 40° C. (e.g., 5° C., 25° C. and/or 40° C.) as compared to a control composition having a pH of 7.2. Methods of assessing antibody aggregation are familiar to one of ordinary skill in the art and include, without limitation, methods such as protein gel electrophoresis and protein staining, Western blotting, mass spectrometry and size-exclusion chromatography. Other methods of assessing antibody aggregation are contemplated herein.

In some embodiments, a composition of the present disclosure has enhanced stability relative to a control composition comprising the chimeric monoclonal antibody (e.g., infliximab) and having a pH of 7.2. Methods of assessing the stability of a composition (e.g., solution) include, without limitation, subjecting the solution to freeze/thaw cycles, agitation (e.g., at 650 rpm for 72 hours at 2 to 8° C.), prolonged temperature storage (e.g., at 2° C. to 8° C., 25° C. and/or 40° C.; and frozen storage (e.g., −20° C. or −70° C.).

In some embodiments, an antibody processing method comprises filtering through a membrane filter a phosphate buffered antibody (e.g., infliximab) solution having a pH of 5.7 to 6.7 and comprising infliximab, thereby producing an antibody concentrate, wherein antibody aggregation in the phosphate buffered antibody solution is reduced relative to a control antibody solution comprising infliximab and having a pH of 7.2. In some embodiments, methods further comprise lyophilizing the antibody concentrate (e.g., for storage). In some embodiments, methods further comprise reconstituting lyophilized antibody in a buffer. In some embodiments, the buffer has a pH of 7.2. In some embodiments the buffer has a pH of less than 7.2 (e.g., a pH of 5.7 to 6.7, e.g., 6.2). In some embodiments, the buffer comprises sucrose and/or surfactant. In some embodiments, sucrose and/or surfactant is added to the buffer after the antibody is reconstituted.

In some embodiments, a composition of the present disclosure is lyophilized. For example, following filtration or purification, a composition may be lyophilized (e.g., for storage). In some embodiments, a lyophilized composition comprises infliximab and sucrose. In some embodiments, a lyophilized composition comprises infliximab and a surfactant. In some embodiments, a lyophilized composition comprises infliximab, sucrose and a surfactant.

### EXAMPLES

### Example 1

The present example describes initial experiments conducted to assess the presence of high molecular weight (HMW) species, primarily in the form of dimers, formed during bioprocessing of a commercial-based infliximab composition. Two prototype bioprocessing runs were conducted (PT-01 and PT02).

In the first run (PT-01), following protein production and initial column-based purification (e.g., at a loading of 177 g/m²) using a phenyl sepharose matrix, column flow-through was concentrated to 20 g/L. The concentrated material was then subjected to diafiltration using 10 diavolume (DV) of 10 mM sodium phosphate, 10% sucrose at pH 7.2. The diafiltered material was further concentrated to 40 g/L. A buffer flush was performed to increase antibody recovery and dilute the antibody solution to a concentration of 25 g/L, corresponding to the concentration of infliximab in a commercial composition. The overall antibody recovery was 92%. Additional samples were collected at predetermined intervals throughout the ultrafiltration/diafiltration (UF/DF) operation to monitor formation of HMW species.

In the second run (PT-02), a smaller membrane (e.g., 0.0088 m² vs. 0.33 m²) was used to evaluate whether increased aggregate formation could be related to production scale. The protein material was otherwise processed using the same operating conditions as described in the first run. The overall product recovery from the second run was 88%, which was comparable to the first run. Again, additional samples were collected at predetermined intervals throughout the UF/DF operation to monitor formation of HMW species.

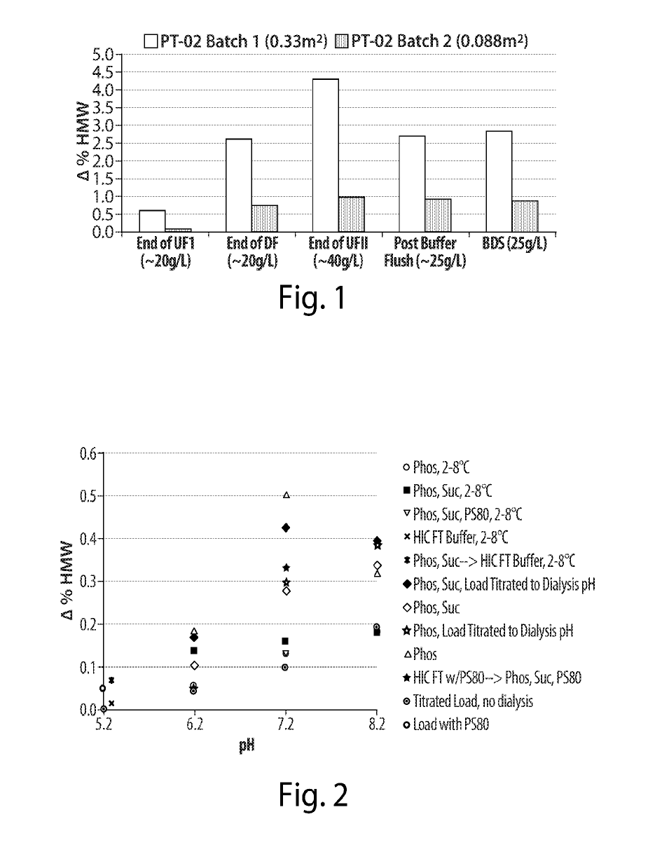

FIG. 1 shows a size exclusion chromatography (SEC) analysis of the collected samples from the first and second bioprocessing runs. For the first run (PT-01), there was a collective change in the percentage of HMW species of 2.8% from loading (“End of UF1”) to collection of the protein product (also referred to herein as biologic drug substance (“BDS”)), with the greatest increase occurring during the diafiltration (DF) (2%) and second ultrafiltration (UF) step (1.7%). For the second run (PT-02), the percentage of HMW species at transition points comparable to the first run were significantly lower for all samples tested.

While the experiments in this Example suggest that bioprocessing scale influences aggregate formation, results presented below demonstrate that the influence is not significant. Regardless of bioprocessing scale, for both prototype bioprocessing runs, the overall change in the percentage of HMW species from loading to collection of the filtered antibody solution (e.g., 0.89%) was greater than the target percentage of less than or equal to 0.5% for the final protein product.

### Example 2

The present example describes experiments conducted to evaluate the impact of buffer pH and composition on high molecular weight (HMW) species formation during antibody bioprocessing. Column flow-through from the PT-02 run described in Example 1 was used for a series of dialysis experiments. In particular, the experiments presented in this Example were for the purpose of examining the effects of changes in pH, presence or absence of sucrose and/or presence or absence of polysorbate 80 (e.g., TWEEN® 80), and temperature.

Results showed an approximately linear increase to a maximum change in the percentage of HMW species of 0.2% as the load (e.g., flow-through) was titrated from pH 5.2 to pH 8.2 (FIG. 2, “Titrated Load, no dialysis).

Similar linear trends were observed when the antibody, in a buffer having a pH of 5.2, was dialyzed directly into buffers ranging from pH 6.2 to 8.2.

Titration of the load prior to dialyzing into a buffer of the same pH did not have a consistent effect on HMW species formation (FIG. 2, “Phos, Load Titrated to Dialysis pH” and “Phos, Suc, Load Titrated to Dialysis pH”).

The presence of sucrose and polysorbate 80 did not demonstrate any significant effect on HMW species formation.

When comparing dialysis results at 2° C. to 8° C. versus ambient temperature, there was an apparent suppression of HMW species formation at colder temperatures.

A follow-up experiment was performed to examine the reversibility of the antibody aggregation by dialyzing column flow-through (FIG. 2, “HIC FT Buffer, 2-8° C.”) first into a buffer having 10 mM sodium phosphate, 10% sucrose and a pH of 7.2, and then dialyzing it back into the column flow-through buffer having a pH of 5.2. This experiment showed that the change in the percentage of HMW species in an antibody solution can be reduced by about 56% (compare 0.160% and 0.071%). Without being bound by theory, it was thought that, at this point in the process, aggregation of the antibody increases when it is close to its isoelectric point (pI) because the isoelectric point of infliximab is approximately 7.5, and the pH of the commercial-based infliximab composition buffer is 7.2. Additional data supported this observation and further showed that the aggregation rate of the antibody was higher at pH 7.2 than at a lower pH.

Based on the results presented in this Example, it was concluded that the commercial-based infliximab composition buffer, having a 10 mM sodium phosphate concentration, a 10% sucrose concentration and a pH of 7.2, is not the most suitable composition buffer for preventing antibody aggregation during bioprocessing of the antibody, possibly due to the proximity of the isoelectric point of infliximab to the composition pH.

The following examples are directed to reducing HMW species formation in an infliximab solution by processing the antibody at a lower pH and colder temperature (relative to the commercial-based bioprocessing composition), prior to making the final pH adjustment.

### Example 3

Based on results obtained in Example 2, a process was developed where the pH of the buffer solutions was maintained at less than or equal to 6.2 for the majority of the diafiltration/ultrafiltration (UF/DF) operation to evaluate the effect of pH on infliximab aggregation during the UF/DF operation. A goal of the experiments presented in this Example was to diafilter infliximab into a phosphate buffer formulated at pH 6.2 having a phosphate concentration of less than the target commercial-based concentration of 10 mM such that a concentrated phosphate solution could be added following filtration to both increase the pH to 7.2 and to reach the target phosphate concentration of 10 mM. This concentrated phosphate solution addition was to be coupled with a concentrated sucrose solution to attain a target sucrose concentration of 10% (using a concentrated 50% sucrose solution).

Initially, titration curves were generated for 10 mM sodium phosphate, with and without 10% sucrose to determine the ratios of monobasic to dibasic sodium phosphate required to target pH 6.2 and 7.2. Based on the ratios, and the 25% additional sucrose solution needed to reach 10% sucrose in the final antibody solution, the concentrations of sodium phosphate, monobasic and dibasic, were defined for the diafiltration buffer (e.g., 4.06 mM monobasic and 0.60 mM dibasic) and the concentrated sucrose solution (e.g., 31.37 mM dibasic). The two buffers were evaluated during a UF/DF operation performed in duplicate using material from PT-02, as described in further detail below in Example 4. The resulting pH of the two buffers was respectively 6.86 and 6.90, and the concentrations of sodium phosphate and sucrose for each were respectively 10 mM and 10%.

The initial target pH for the final infliximab solution was 7.2±0.3, based on the commercial-based composition, therefore the final infliximab solution from both runs were acceptable; however, additional experiments (below) were performed to adjust the pH to 7.2. The following experiments were used to test whether (1) the ratios of monobasic and dibasic sodium phosphate in the infliximab solution could be modified to target pH 7.5 based on the titration curves generated previously above, and (2) whether the UF/DF starting material (e.g., phenyl sepharose flow-through having a pH of 5.2) could be titrated to the target diafiltration pH of 6.2 prior to starting the UF/DF operation.

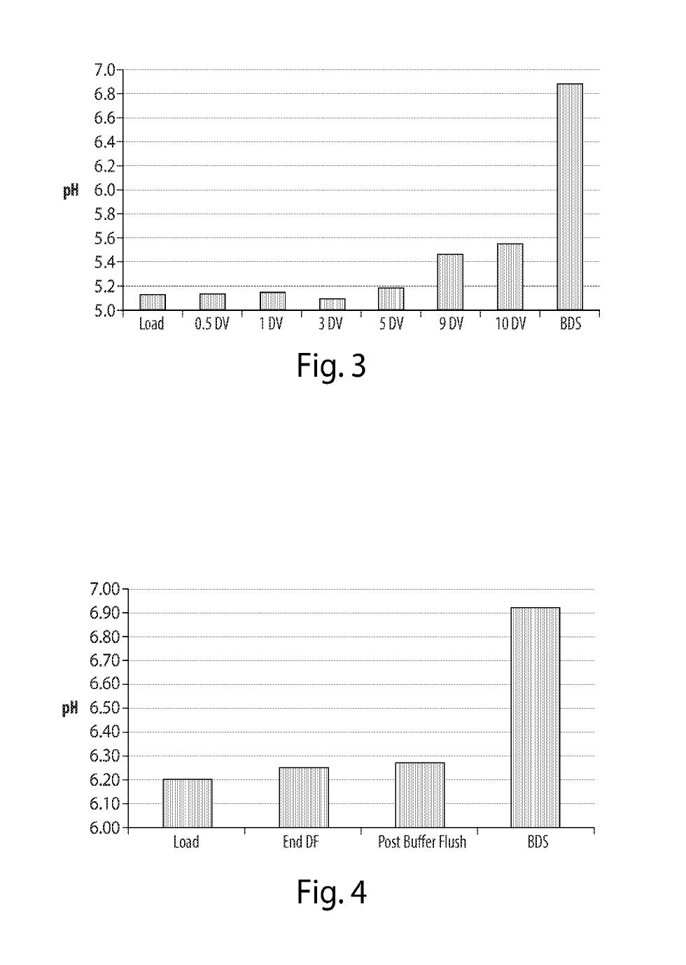

The following experiments test whether the ratios of monobasic and dibasic sodium phosphate in the infliximab solution could be modified to target pH 7.5. Based on the titration curve calculations, the concentrations of sodium phosphate, monobasic and dibasic, were defined for the modified diafiltration buffer (e.g., 2.54 mM monobasic and 0.37 mM dibasic) and the modified concentrated sucrose solution (e.g., 38.42 mM dibasic). To increase the ratio of dibasic to monobasic sodium phosphate in the final infliximab solution, the total amount of phosphate in the diafiltration buffer was reduced from 4.66 mM to 2.91 mM. The modified buffers were evaluated during a UF/DF operation performed using material from a third prototype bioprocessing run (PT-03), while monitoring the pH at appropriate intervals throughout the process. The pH at each interval is shown in FIG. 3. The resulting pH of the final infliximab solution was 6.89, and the concentrations of sodium phosphate and sucrose were respectively 10 mM and 10%. Based on the minimal pH transition observed during the diafiltration step (the diafiltration buffer was formulated at pH 6.2) and the reduction in overall phosphate concentration of the diafiltration buffer, it was thought, without being bound by theory, that there was not sufficient buffering capacity in the diafiltration buffer to affect the expected pH transition from pH 5.2 to pH 6.2, thus preventing a “spike” in pH of the antibody solution to the target of 7.2 by addition of the concentrated sucrose solution.

The following experiments test whether the UF/DF starting material (e.g., phenyl sepharose flow-through having a pH of 5.2) could be titrated to the target diafiltration pH of 6.2 prior to starting the UF/DF operation. The concentrations of sodium phosphate, monobasic and dibasic, as defined above, were used for the diafiltration buffer (e.g., 4.06 mM monobasic and 0.60 mM dibasic) and concentrated sucrose buffer (e.g., 31.37 mM dibasic). The buffers were evaluated during a UF/DF operation performed using a second batch of material from the PT-03 bioprocessing run, while again monitoring the pH at appropriate intervals throughout the process. The pH at these intervals is shown in FIG. 4. The resulting pH of the final infliximab solution was 6.92, and the concentrations of sodium phosphate and sucrose were respectively 10 mM and 10%. Based on these results, it was thought, without being bound by theory, that although the load was titrated to pH 6.2, the diafiltration buffer did not have sufficient ionic strength to fully displace the ions remaining from the phenyl sepharose flow-through buffer and that the concentrated sucrose buffer did not have sufficient buffering capacity to affect the necessary change in pH after diafiltration. Example 5, below, describes a fourth prototype bioprocessing run (PT-04) to test whether tribasic sodium phosphate could be used in the concentrated sucrose buffer to reach the target pH of 7.2 of the final infliximab solution.

### Example 4

Two additional UF/DF operations at different temperatures, one “cold” and one “ambient,” were performed using material generated during the second prototype bioprocessing run (PT-02) to evaluate the initial buffer combination described in Example 3, as well as to test whether HMW species formation could be reduced by reducing the processing temperature. With the exception of process temperature, the conditions used in both runs were the same. The target mass loading was 180 g/m². For the “cold” UF/DF run, temperature was maintained using a chiller recirculating water through a jacketed vessel and a heat exchanger attached to the retentate side of the UF/DF cassette holder. The chiller was maintained at 2° C., while the water bath within the jacketed vessel was measured at 4° C. to 6° C. by thermometer. The load material, phenyl sepharose flow-through, was concentrated to a target of 40 g/L, followed by diafiltration that included 10 diavolumes of a 4.7 mM sodium phosphate buffer having a pH of 6.2. After a buffer flush to improve recovery and dilute the antibody pools to 32 g/L, the antibody recovery was measured at 92% for the cold run and 104% for the ambient run.

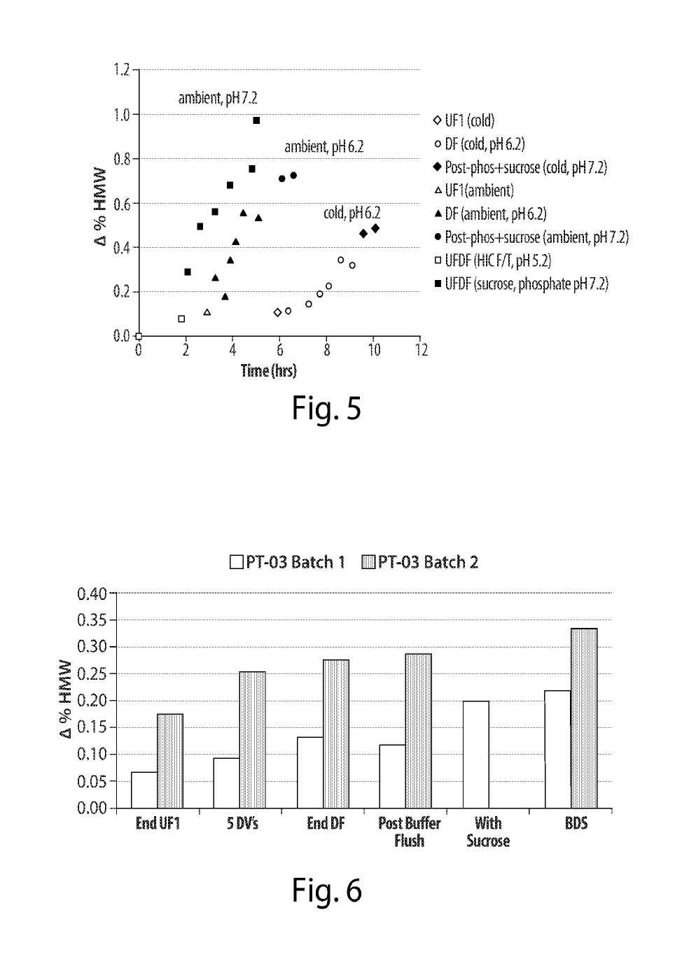

As expected, the permeate flux was significantly lower for the cold UF/DF run relative to the ambient run. A 25% addition of concentrated sucrose buffer (e.g., 31.4 mM sodium phosphate, 50% sucrose) diluted the pools to approximately 25 g/L and brought the sucrose concentration to 10%. Samples were removed periodically throughout each UF/DF run to be analyzed by size exclusion chromatography (SEC). FIG. 5 shows the results of this analysis, compared to the first small scale UF/DF run from PT-02, performed using a buffer having a 10 mM sodium phosphate buffer concentration, a 10% sucrose concentration and a pH of 7.2. There was an apparent reduction in HMW species formation when diafiltration was performed using a buffer having a pH 6.2 without sucrose versus using a buffer having a pH of 7.2 with sucrose. There was a further reduction in HMW species formation of 0.25% when the UF/DF was performed at 4° C. to 6° C. There did not appear to be a direct correlation between processing time and HMW species formation. There was a general increase in HMW species formation relative to processing time, as shown in FIG. 5; however, that increase occurred during the transition to use a buffer having a 10 mM sodium phosphate concentration.

### Example 5

The present example describes further experiments conducted to evaluate HMW species formation using the buffer combinations described in Example 3 using infliximab from the third prototype bioprocessing run (PT-03). In particular the first UF/DF operation experiment was for the purpose of evaluating HMW species formation when using a concentrated sucrose buffer with a higher targeted pH. With the exception of the process temperature, which was maintained between 5° C. and 8° C., the conditions used were the same as in previous examples. Initially, a mass loading of 235 g/m² was targeted; however, the loading was reduced to 180 g/m² due to the decline in flux observed during the concentration step from about 99 to 15 LMH and the reduced load concentration. The decreased flux and load concentration combined to result in an increased processing time for the batch. The antibody from the phenyl sepharose flow-through starting material, was concentrated to 40 g/L, followed by a diafiltration step consisting of 10 diavolumes of a sodium phosphate buffer at pH 6.2 (e.g., 2.9 mM Sodium Phosphate). After a buffer flush to improve recovery and dilute the antibody concentration to 32 g/L, the recovery of the antibody was 97%. A concentrated sucrose buffer was added (e.g., 25% addition of 38.4 mM sodium phosphate 50% sucrose) to adjust the concentration of sodium phosphate, dilute the antibody to a concentration of 27 g/L and achieve a sucrose concentration to 10%. Polysorbate-80 was added to a concentration of 0.01% prior to filtration of the final infliximab solution. Samples were collected at various stages during the UF/DF operation and analyzed for HMW species formation by SEC, as shown in FIG. 6 (PT-03 Batch 1). An increase in HMW species formation of 0.22% was observed between the load (phenyl sepharose flow-through) and the final infliximab solution. There was a 0.08% increase in HMW species formation during the addition of the concentrated sucrose buffer and pH adjustment from pH 5.6 to 6.9 (see FIG. 4). These results confirmed previous observations of HMW species formation during this pH transition.

A second UF/DF run was performed using infliximab material from PT-03, with a goal of addressing HMW species formation during the pH transition issue, as described in Example 3. With the exception of the process temperature, which was maintained between 10° C. and 11° C., the conditions used were the same as in previous examples. A target mass loading of 90 g/m² was used to reduce processing time for this experiment, as the primary objective was evaluating the pH transition. The phenyl sepharose flow-through starting material was first titrated to pH 6.2, for example, by a 1.23% addition of 0.9M Bis-Tris, 0.24M Tris Base. There was no apparent change in percentage of HMW species observed during the titration of the load for this run. The titrated antibody solution (load) was concentrated to an antibody concentration of 40 g/L, followed by diafiltration consisting of 10 diavolumes of a sodium phosphate buffer (e.g., 4.7mM Sodium Phosphate, pH 6.2). After a buffer flush to improve recovery and dilute the antibody concentration to 32 g/L, the recovery was measured at 77%. The lower recovery may have been due to losses associated with the low recirculation volume of the buffer and/or the hold-up volume in the heat exchanger. The antibody concentration was further diluted to 27 g/L and a sucrose concentration of 10% was achieved with the addition of a concentrated sucrose buffer (e.g., 25% addition of 31.4 mM sodium phosphate, 50% sucrose). Samples were collected during various stages of the UF/DF operation and were analyzed for HMW species formation by SEC, as presented in FIG. 6 (PT-03 Batch 2). An increase in HMW species of 0.33% was observed between the load and final infliximab solution. These results and the apparent utility of tribasic phosphate in the concentrated sucrose buffer indicated it is possible to remove the load titration step to minimize processing time at pH 6.2.

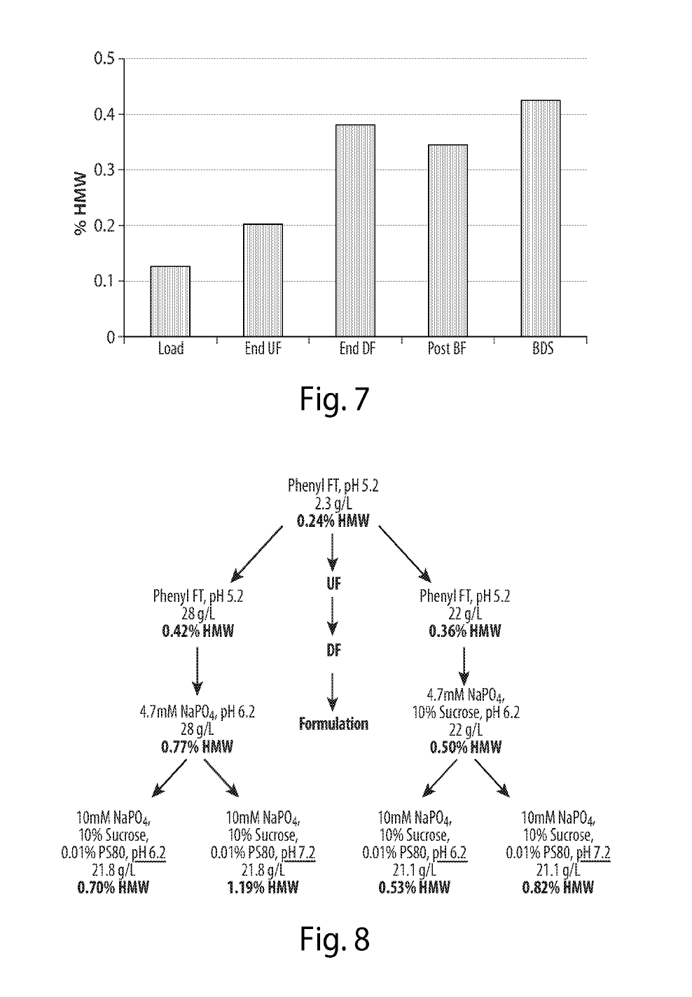

Additional experiments were performed using infliximab material from a fourth prototype bioprocessing run (PT-04) with the goal of evaluating the use of tribasic sodium phosphate in the concentrated sucrose buffer conditions as was initially developed using infliximab material from PT-03. With the exception of the process temperature, which was maintained between 6° C. and 10° C., the conditions used were the same as in previous examples. The target mass loading was 180 g/m². The antibody from the phenyl sepharose flow-through starting material was concentrated to 40 g/L, followed by a diafiltration consisting of 10 diavolumes of a sodium phosphate buffer at pH 6.2 (e.g., 4.7 mM sodium phosphate consisting of 4.06 mM monobasic and 0.60 mM dibasic sodium phosphate). After a buffer flush to improve recovery and dilute antibody to a concentration of 32 g/L, the antibody recovery was 95%. The concentrated sucrose buffer was added (e.g., 25% addition of 31.4 mM sodium phosphate (12.01 mM monobasic, 1.77 mM dibasic, 17.58 mM tribasic), 50% sucrose) to dilute the antibody concentration to 26 g/L and achieve a sucrose concentration of 10%. Polysorbate-80 was added to a concentration of 0.01% prior to filtration of the final infliximab solution. The final pH after addition of both the concentrated sucrose buffer and the polysorbate-80 was 7.05. A portion of the antibody solution was retained after the buffer flush and prior to addition of the concentrated sucrose buffer for further pH development. The results indicated that addition of a concentrated sucrose buffer containing 31.33 mM sodium phosphate (e.g., 10.70 mM monobasic, 1.62 mM dibasic, 19.01 mM tribasic) resulted in a final pH of 7.18. Samples were collected at various stages throughout the UF/DF operation and were analyzed for HMW species formation by SEC, as shown in FIG. 7. An increase in HMW species formation of 0.30% was observed between the load and final infliximab solution. There was a 0.08% increase in HMW species formation during addition of the concentrated sucrose buffer and pH adjustment from pH 5.6 to 6.9 (FIG. 7), as also observed in previous examples.

### Example 6

The present example describes experiments with the goal of evaluating HMW species formation in additional bioprocessing preparations of infliximab and whether addition of a concentrated sucrose buffer and different pH (e.g., pH 6.2 and 7.2) of the antibody solution would affect HMW species formation. For these experiments, the antibody concentration was reduced to from 25 g/L to 20 g/L and the target concentrations during ultrafiltration were scaled accordingly. Experiments were performed at temperatures between 2° C. and12° C.

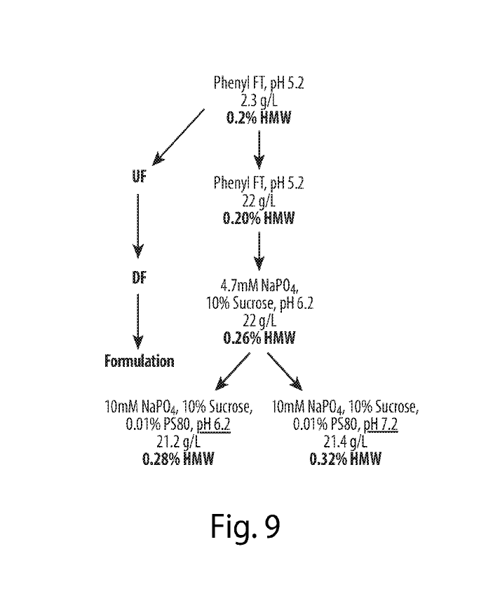

As shown in FIG. 8, addition of 10% sucrose to the diafiltration buffer improved (reduced) the HMW species formation throughout the operation. It is also possible that the reduced diafiltration concentration may have also contributed to the reduced HMW species formation after ultrafiltration. By reducing the pH from 7.2 to 6.2, the initial HMW species target of the commercial-based infliximab antibody composition was achieved regardless of whether sucrose was present in the diafiltration buffer. This experiment was repeated with sucrose in the diafiltration buffer, to confirm these results, as shown in FIG. 9.

The data from these experiments confirmed that the presence of sucrose in the diafiltration buffer reduced HMW species formation during the UF/DF operation. Furthermore, the final infliximab solution containing 10 mM sodium phosphate, 10% sucrose at pH 6.2 allowed for lower antibody concentration (22 g/L) during diafiltration, which also aided in reducing HMW species formation.

### Example 7

Following development of the UF/DF and buffer conditions for reducing HMW species formation, additional experiments were performed to examine antibody stability in the buffer conditions. Infliximab material was dialyzed into 10 mM sodium phosphate at various pH (5.7, 6.0 and 6.7). The resulting pH of each of the antibody solutions was 5.7, 6.0 and 6.4, respectively. Each of the antibody solutions was evaluated for antibody stability in various conditions and compared to the stability of a commercial-based infliximab antibody composition in the commercial composition at pH 7.2.

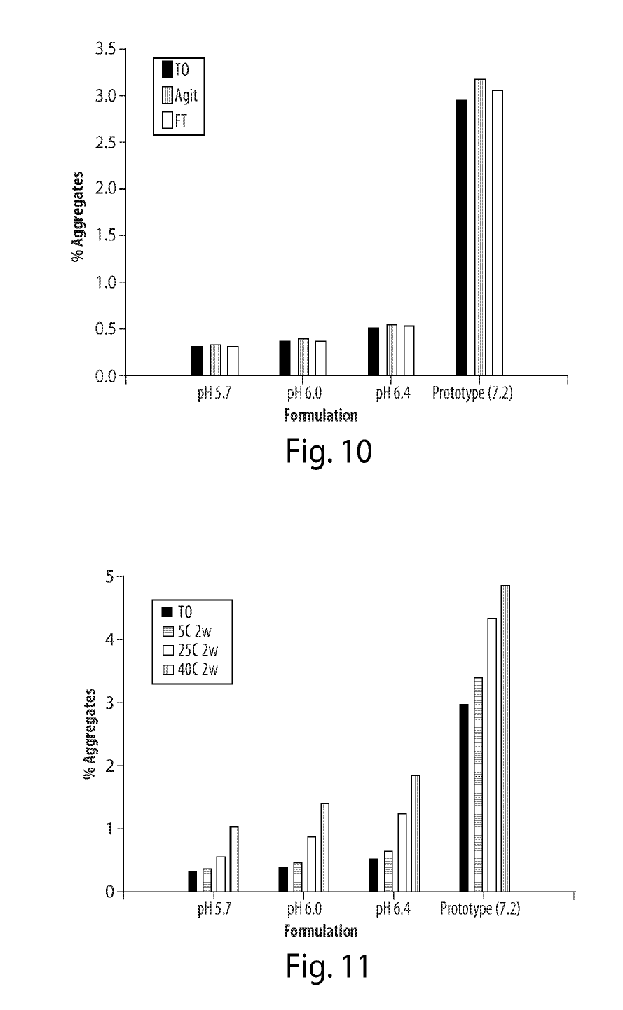

The percentage of HMW species formed in each of the antibody solutions was assessed by SEC prior to and after each of the following conditions: 5 freeze/thaw cycles (FIG. 10); agitation at 650rpm for 72 hours at 2-8° C. (FIG. 10); incubation at 5° C., 25° , or 40° C. (FIG. 11, Table 1); and storage at −20° C. or −70° C. (Table 1). The percent purity of the antibody solutions under reducing conditions was also assessed following incubation at 5° C., 25° , or 40° C. (Table 2); and storage at −20° C. or −70° C. (Table 2).

In each of the conditions tested, the new buffer conditions resulted in improved aggregation profiles (reduced formation of HMW species) for the infliximab solutions.

TABLE 1
Size exclusion chromatography results for
frozen and accelerated storage stability.

Time point, Months

| Temp | Sample | 0 | 0.5 | 1 | 3 | 6 |
|---|---|---|---|---|---|---|
| 2-8 C. | pH 5.7 | 0.3 | 0.4 | 0.4 | 0.5 | 0.5 |
| | pH 6.0 | 0.4 | 0.5 | 0.5 | 0.6 | 0.8 |
| | pH 6.4 | 0.5 | 0.6 | 0.7 | 0.9 | 1.0 |
| 25 C. | pH 5.7 | 0.3 | 0.6 | 0.6 | * | * |
| | pH 6.0 | 0.4 | 0.9 | 1.0 | * | * |
| | pH 6.4 | 0.5 | 1.2 | 1.3 | * | * |
| 40 C. | pH 5.7 | 0.3 | 1.0 | 1.3 | * | * |
| | pH 6.0 | 0.4 | 1.4 | 1.8 | * | * |
| | pH 6.4 | 0.5 | 1.8 | 2.2 | * | * |
| −20 C. | pH 5.7 | 0.3 | * | 0.3 | 0.3 | 0.3 |
| | pH 6.0 | 0.4 | * | 0.3 | 0.3 | 0.3 |
| | pH 6.4 | 0.5 | * | 0.4 | 0.5 | 0.4 |
| −70 C. | pH 5.7 | 0.3 | * | 0.3 | 0.3 | 0.3 |
| | pH 6.0 | 0.4 | * | 0.3 | 0.3 | 0.3 |
| | pH 6.4 | 0.5 | * | 0.4 | 0.5 | 0.4 |

* = Not Tested

TABLE 2
Percent purity (reduced) results for
frozen and accelerated stability

| | | Time point, Months | | | |
|---|---|---|---|---|---|
| Temp | Sample | 0 | 0.5 | 1 | 6 |
| 5 C. | pH 5.7 | 99.5 | 98.5 | 96.9 | 99.5 |
| | pH 6.0 | 99.5 | 98.4 | 97.3 | 99.5 |
| | pH 6.4 | 97.5 | 98.6 | 97.4 | 99.5 |
| 25 C. | pH 5.7 | 99.5 | 98.3 | 97.6 | * |
| | pH 6.0 | 99.5 | 98.3 | 97.6 | * |
| | pH 6.4 | 97.5 | 98.3 | 97.4 | * |
| 40 C. | pH 5.7 | 99.5 | 97.5 | 96.0 | * |
| | pH 6.0 | 99.5 | 96.9 | 95.4 | * |
| | pH 6.4 | 97.5 | 96.0 | 94.3 | * |
| −20 C. | pH 5.7 | 99.5 | * | 97.9 | 99.6 |
| | pH 6.0 | 99.5 | * | 97.8 | 99.6 |
| | pH 6.4 | 97.5 | * | 97.8 | 99.5 |
| −70 C. | pH 5.7 | 99.5 | * | 97.8 | 99.7 |
| | pH 6.0 | 99.5 | * | 97.9 | 99.6 |
| | pH 6.4 | 97.5 | * | 97.8 | 99.5 |

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An antibody processing method, comprising filtering through a membrane filter a phosphate buffered antibody solution having a pH of 5.7 to 6.7 and comprising infliximab, thereby producing an antibody concentrate, wherein antibody aggregation in the phosphate buffered antibody solution is reduced relative to a control antibody solution comprising infliximab and having a pH of 7.2.

2. The method of claim 1, wherein the antibody solution has a pH of 6.0 to 6.5.

3. The method of claim 2, wherein the antibody solution has a pH of 6.5.

4. The method of claim 2, wherein the antibody solution has a pH of 6.4.

5. The method of claim 2, wherein the antibody solution has a pH of 6.3.

6. The method of claim 2, wherein the antibody solution has a pH of 6.2.

7. The method of claim 2, wherein the antibody solution has a pH of 6.1.

8. The method of claim 2, wherein the antibody solution has a pH of 6.0.

9. The method of any one of claims 1-8, wherein the antibody solution comprises sodium phosphate.

10. The method of claim 9, wherein the antibody solution comprises sodium phosphate at a concentration of 5 mM to 15 mM.

11. The method of claim 10, wherein the antibody solution comprises sodium phosphate at a concentration of 10 mM.

12. The method of any one of claims 1-11, wherein the antibody solution comprises sucrose.

13. The method of claim 12, wherein the antibody solution comprises sucrose at a concentration of 5% to 15% (w/v).

14. The method of claim 13, wherein the antibody solution comprises sucrose at a concentration of 10% (w/v).

15. The method of any one of claims 1-14, wherein the antibody solution comprises a surfactant.

16. The method of claim 15, wherein the antibody solution comprises surfactant at a concentration of 0.005% to 0.015% (w/v).

17. The method of claim 16, wherein the antibody solution comprises surfactant at a concentration of 0.01% (w/v).

18. The method of any one of claims 15-17, wherein the surfactant is polysorbate 80.

19. The method of any one of claims 1-18, wherein the antibody solution comprises infliximab at a concentration of 15 g/L to 100 g/L.

20. The method of claim 19, wherein the antibody solution comprises infliximab at a concentration of 20 g/L to 60 g/L.

21. The method of any one of claims 1-20, wherein the temperature of the antibody solution is 2° C. to 12° C.

22. The method of claim 21, wherein the temperature of the antibody solution is 4° C. to 6° C.

23. The method of any one of claims 1-22, wherein the membrane filter has a pore size of 0.1 to 0.001 micron.

24. The method of any one of claims 1-23, wherein the antibody solution is filtered through the membrane filter by tangential flow filtration.

25. The method of any one of claims 1-24 further comprising collecting the antibody concentrate.

26. A composition comprising an antibody solution comprising infliximab and having a pH of 5.7 to 6.7.

27. The composition of claim 26, wherein the antibody solution has a pH of 6.0 to 6.5.

28. The composition of claim 27, wherein the antibody solution has a pH of 6.5.

29. The composition of claim 27, wherein the antibody solution has a pH of 6.4.

30. The composition of claim 27, wherein the antibody solution has a pH of 6.3.

31. The composition of claim 27, wherein the antibody solution has a pH of 6.2.

32. The composition of claim 27, wherein the antibody solution has a pH of 6.1.

33. The composition of claim 27, wherein the antibody solution has a pH of 6.0.

34. The composition of any one of claims 26-33, wherein the antibody solution comprises sodium phosphate.

35. The composition of claim 34, wherein the antibody solution comprises sodium phosphate at a concentration of 5 mM to 15 mM.

36. The composition of claim 35, wherein the antibody solution comprises sodium phosphate at a concentration of 10 mM.

37. The composition of any one of claims 26-36, wherein the antibody solution comprises sucrose.

38. The composition of claim 37, wherein the antibody solution comprises sucrose at a concentration of 5% to 15% (w/v).

39. The composition of claim 38, wherein the antibody solution comprises sucrose at a concentration of 10% (w/v).

40. The composition of any one of claims 26-39, wherein the antibody solution comprises a surfactant.

41. The composition of claim 40, wherein the antibody solution comprises surfactant at a concentration of 0.005% to 0.015% (w/v).

42. The composition of claim 41, wherein the antibody solution comprises surfactant at a concentration of 0.01% (w/v).

43. The composition of any one of claims 40-42, wherein the surfactant is polysorbate 80.

44. The composition of any one of claims 26-43, wherein the antibody solution comprises the chimeric monoclonal antibody at a concentration of 15 g/L to 100 g/L.

45. The composition of claim 44, wherein the antibody solution comprises the chimeric monoclonal antibody at a concentration of 20 g/L to 60g/L.

46. The composition of any one of claims 26-45, wherein the infliximab is more stable when the composition is stored at temperatures of −70° C. to at least 40° C. relative to a control composition having a pH of 7.2.
